(19) United States
(12) Patent Application Publication
(10) Pub. No.: US 2009/0137992 A1
(43) Pub. Date: May 28, 2009

(54) Apparatus and Method for Treating Glaucoma

(76) Inventor: Ravi Nallakrishnan, Westmont, IL (US)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/946,021

(22) Filed: Nov. 27, 2007

(51) Int. Cl. A61B 18/18; A61M 35/00

(52) U.S. Cl. 606/6; 604/294

(57) ABSTRACT

An irrigating probe for providing a flow of liquid to the trabecular meshwork of the eye has a first, guide prong that contacts the cornea to position a second, irrigator prong proximate the trabecular meshwork. The irrigator prong has at least one outlet port communicating with an internal flow channel which, in turn, communicates with a liquid supply providing a steady or a pulsed flow.

### FIELD OF THE INVENTION

The present invention is generally directed to a surgical treatment for glaucoma and, more particularly, to apparatus and methods for use of the apparatus to treat glaucoma by reducing the intraocular pressure within the eye by clearing blockages and/or obstructions from the trabecular meshwork.

### BACKGROUND OF THE INVENTION

Glaucoma is a disorder of the optic nerve causing loss of both central and peripheral vision that usually occurs due to elevated intraocular pressure within the eye. The disease and its treatments are well-described in the prior art and portions of such descriptions are included herein as drawn from U.S. Pat. Nos. 6,827,700 and 4,985,417.

Glaucoma often goes undiagnosed because the increase in intraocular pressure is not accompanied by pain or discomfort and may go undetected until the increased pressure results in damage to the optic nerve.

In a healthy eye, an average intraocular pressure can range from about 15.5 mm Hg and 20.5 mm Hg. Readings above 20.5 mm indicate the possibility of glaucoma. Pressures above 30 mm Hg are almost definitely pathological in nature.

The eye is a hollow structure that contains a clear fluid called “vitreous humor” formed in the posterior chamber of the eye by the ciliary body at a rate of about 2.5 microliters per minute. This fluid then passes around the lens, through the pupillary opening in the iris and into the anterior chamber of the eye. Once in the anterior chamber, the fluid drains out of the eye through two different routes. In the “uveoscleral route” the fluid percolates between muscle fibers of the ciliary body. This route accounts for approximately 10 percent of the aqueous outflow in humans.

The primary pathway for aqueous outflow in humans is through the “canalicular” route that involves the trabecular meshwork and Schlemm's canal.

The trabecular meshwork and Schlemm's canal are located at the junction between the iris and the sclera. This junction or corner is called “the angle.” The trabecular meshwork is wedge shaped in structure and runs around the entire circumference of the eye, forming a three-dimensional sieve structure. The meshwork is formed of collagen beams which are aligned with a monolayer of cells called the trabecular cells. The spaces between the collagen beams are filled with an extracellular substance that is produced by the trabecular cells. These cells also produce enzymes that degrade the extracellular material. Schlemm's canal is adjacent to the trabecular meshwork and the outer wall of the trabecular meshwork coincides with the inner wall of Schlemm's canal. Schlemm's canal is a tube-like structure that also extends around the circumference of the cornea. In human adults, Schlemm's canal is believed to be divided by septa into a series of autonomous, dead-end canals.

The aqueous fluid travels through the spaces between the trabecular meshwork across the inner wall of Schlemm's canal and into the canal through a series of about 25 collecting channels that drain from Schlemm's canal into the episcleral venous system. In a normal situation, aqueous production is equal to aqueous outflow and interoccular pressure remains fairly constant. In glaucoma, the resistance through the canicular outflow system is abnormally high.

Glaucoma can be considered as primary and secondary, primary glaucoma being either congenital or capable of developing later in life, the adult form of glaucoma can be caused by angle closure, angle obstruction or resistance to outflow known as chronic simple glaucoma. Acute angle closure glaucoma results in red painful eyes, an overt indication that some abnormality exits. In the glaucoma condition termed as chronic simple glaucoma, however, the eyes appear normal and such conditions can go undiagnosed for along period of time.

In primary open angle glaucoma, which is the most common type of glaucoma, the abnormal resistance is believed to be along the outer aspect of the trabecular meshwork and the inner wall of Schlemm's canal. It is believed that an abnormal metabolism of the trabecular cells leads to an excess of buildup of extracellular materials or a buildup of abnormally stiff materials in this area. Other forms of glaucoma, such as angle closure glaucoma and secondary glaucoma, also involve decreased outflow through the canalicular pathway, but the increased resistance is from other causes such as mechanical blockage, inflammatory debris, cellular blockages, and the like.

Intraocular pressure builds up because the aqueous fluid cannot exit the eye fast enough. As the fluid builds up, the intraocular pressure within the eye increases and compresses the axons on the optic nerve which also may compromise the vascular supply to the optic nerve.

The clinical treatment of glaucoma is approached in a stepwise fashion. Medication is often the first treatment option. Administered either topically or orally, these medications work to either reduce aqueous production or they act to increase outflow. Currently available medications have many serious side effects including congestive heart failure, respiratory distress, hypertension, depression, renal stones, aplastic anemia, sexual dysfunction and death.

In addition, medication is often ineffective where patients do not follow the prescribed regimen of taking the medication. In cases where medication does not control the condition, surgical means must be employed. One surgical procedure commonly used is laser trabeculoplasty, a procedure in which thermal energy provided by a laser is applied to a number of non-contiguous spots in the trabecular meshwork. It is believed that the laser energy in some way stimulates the metabolism of the trabecular cells to change the extracellular material in the trabecular meshwork. In approximately 80 percent of the cases, aqueous outflow is enhanced and pressure decreases. This effect is often not long lasting and about half of the patients treated by this method experience increased pressure over a period of five years following the procedure.

Use of a laser beam is thought to displace protein from the trabecular meshwork and to stimulate the autoimmune system at the treated site to digest the displaced protein fragments.

Laser treatment has the advantage of being non-invasive but, to date, also exhibits an increase in IOP over time which will require monitoring and probable future treatment.

Another procedure typically used is forming a hole in the sclera and angle region which provides a route allowing the fluid to leave the eye.

The most common surgical procedure for reducing eye pressure is a trabeculotomy, a procedure in which an incision is made in the conjunctiva, the transparent tissue that covers the sclera and a section of the trabecular meshwork is removed. This forms a hole though which the aqueous fluid can flow to relieve the elevated pressure. This also leaves an open pathway for infection.

Apparatus and methods of treating glaucoma are well-represented in the prior art.

U.S. Patent Application Publication US2006/0173446 (Dacquay, et al.) and European Patent Application EP1 685 815 (Dacquay, et al.) teaches and describes a surgical apparatus for introducing pulses of liquid to perforate or stimulate the trabecular network. The device consists of a single nozzle extending from a hand piece having internal tube which heated irrigating solution is passed, and a fiber optic as a light source. This apparatus lacks a feature that aligns the nozzle of the meshwork other than the surgeon's hand.

U.S. Pat. No. 7,094,225 (Tu et al) and U.S. Pat. No. 7,273,475 (Tu et al) teach and describe a medical device and methods of use for glaucoma treatment. The '475 patent is a continuation of the '225 patent. The '225 patent teaches the placement of a shunt providing mechanical drain for excess fluid while the '475 patent teaches and claims the method of applying a vacuum to suck fluid through the trabecular meshworkwork.

U.S. Pat. No. 4,985,417 (Trager et al) teaches and describes a treatment of glaucoma by providing a chemical compound to act on the fibers of the trabecular meshwork.

U.S. Patent Application Publication US2003/0120200 (Bergheim et al) teaches and describes an apparatus and method for treating glaucoma by placing an implant through the trabecular meshwork work to act as a drain.

U.S. Pat. No. 7,041,114 (Dan) teaches and describes a surgical tool and method for extracting tissue from wall of an organ which allows a surgeon to manually and mechanically cut an opening in the trabecular meshwork.

U.S. Patent Application Publication 2004/0186534 (Shadduck) teaches and describes devices and techniques for treating glaucoma by using microimplantable bodies in the trabecular meshwork as a target for irradiation.

U.S. Pat. No. 7,282,046 (Simon) teaches and describes a glaucoma treatment method using a laser to stimulate the ciliary region of the eye to ablate debris.

U.S. Pat. No. 6,827,700 (Lynch, et al.) teaches and describes a surgically-implanted shunt to provide a flow path for the aqueous fluid to exit the anterior chamber and keep IOP at acceptable levels.

Irrigating the trabecular meshwork reduces the IOP without requiring the cutting away of portions of the trabecular meshwork. This procedure has been carried out with equipment typically used for procedures such as phacoemulsification, using ultrasound energy provided by a hollow vibratory needle which may also provide a pathway for aspiration of the irrigating fluid. The use of a non-vibratory instrument with a guide to aid in directing the irrigating fluid to the trabecular meshwork finds particular utility.

### BRIEF DESCRIPTION OF THE DRAWINGS

These and further features of the present invention can be better understood through reference to the accompanying drawings, in which:

FIG. 1 illustrates the physiology of the eye in cross-section;

FIG. 2 is a second cross-section showing the flow of aqueous fluid;

FIG. 3 is a perspective view of a prior art trabeculotomy probe with a guide;

FIGS. 4A and 4B are drawings illustrating a prior art surgical technique;

FIG. 5 is a perspective view partially in section of an irrigating probe;

FIG. 6 is a view along 6-6 of FIG. 5;

FIG. 7 is a bottom perspective view showing the irrigating liquid flow path; and

FIG. 8 is a sectional view taken along line 8-8 of FIG. 5.

### DETAILED DESCRIPTION OF THE DRAWINGS

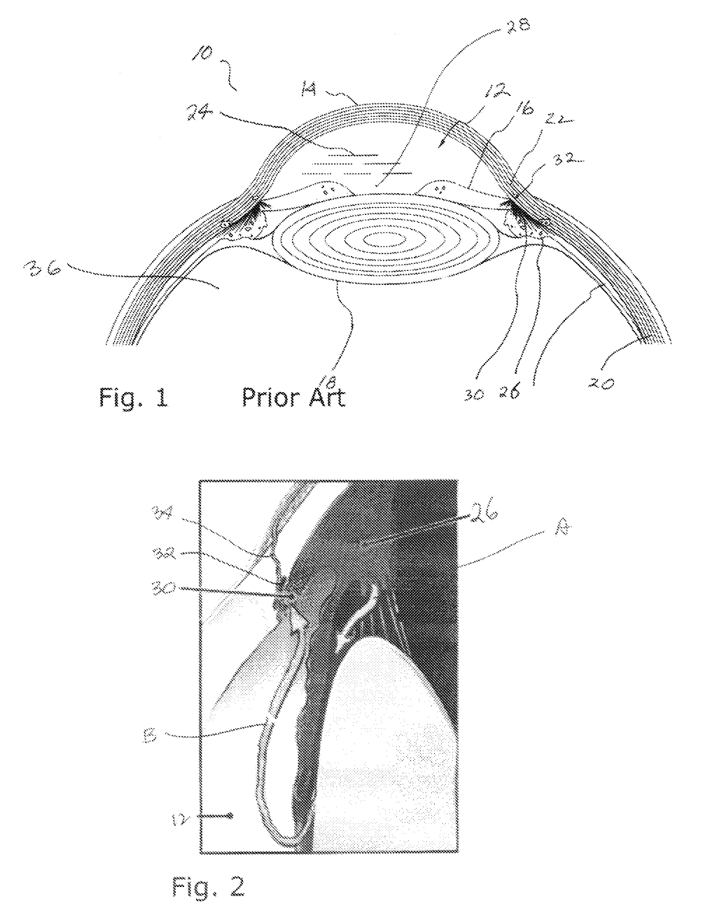

Referring now to FIG. 1, the numeral 10 indicates generally a cross-sectional view of the eye. This figure is a prior art illustration appearing as FIG. 9 of U.S. Pat. No. 5,738,677.

Anterior chamber 12 is generally defined by transparent cornea 14, iris 16, lens capsule 18 and sclera 20 which merges with cornea 14 at limbus 22. Chamber 12 is filled with aqueous vitreous humor (AVH) 24 in sufficient supply to keep chamber 12 full.

As seen in FIGS. 1 and 2, AVH 24 is produced by the ciliary body 26 and enters chamber 12 via flow path portion A, past lens capsule 18 and through cornea 28 along flow path portion B. Because AVH 24 is continuously produced, it must be drained from chamber 12 to prevent a dangerous buildup of intraocular pressure (IOP) with the attendant onset of glaucoma.

A structure for draining aqueous fluid from chamber 12 consist of the trabecular meshwork 30, extending around the periphery of eye 10 beneath sclera 20. AVH 12 under pressure flows through trabecular meshwork 30 and enters Schlemm's canal 32 which then directs the fluid to collecting channels 34. Under normal circumstances trabecular meshwork 30 provides sufficient void volume to accommodate the outflow of AVH 24 and keep the IOP at safe levels. This system is disrupted when trabecular meshwork 30 becomes damaged or clogged.

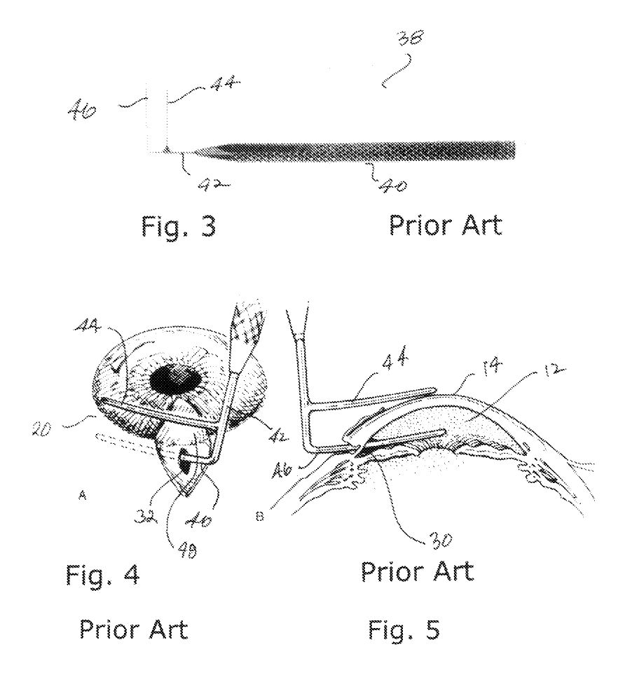

Referring now to FIG. 3, numeral 38 identifies a prior art surgical instrument manufactured by ASICO LLC as it model AE-1630 (right-hand curve) and AE-1631 (left-hand curve) Harms Trabeculotomy Probe with Guide. Probe 38 has a handle 40 with a shaft 42 extending therefrom. A pair of curved and parallel prongs 44, 46 are formed integrally with shaft 42, with upper prong 44 acting as a guide while lower prong 46 is used in a trabeculotomy procedure to tear a hole in the trabecular meshwork 30. Prongs 44, 46 are curved to follow the curvature of the eye and Schlemm's canal and are provided with both left- and right-hand curves.

FIGS. 4A and 4B are taken from page 251 of Manual of Glaucoma Diagnosis and Management, Theodore Krupin, et al, Churchill Livingstone 1988. In FIG. 4A, a scleral incision has been made whereby scleral flap 48 is folded back to reveal Schlemm's canal 32. Lower prong is inserted into Schlemm's canal 30 as shown with guide 44 positioned above sclera 20.

As shown in FIG. 4B, probe 38 is then rotated to bring guide prong 44 into contact with cornea 14 and to force lower prong 46 through trabecular meshwork 30 into chamber 12, creating a passageway for AVH 24 to pass into Schlemm's canal 32. Thereafter, flap 48 is sutured back in place.

The present invention provides methods and apparatus for restoring the flow of AVH 24 without requiring the tearing or cutting of trabecular meshwork 30.

For patients exhibiting increased IOP found to be uncontrollable by either topical or systemic medications were treated use of a surgical procedure to effect improved flow through the trabecular meshwork is warranted.

Within the scope of the invention, irrigation of the trabecular meshwork 30 can be accomplished in a number of ways. One method is to use an instrument that produces a pulsed flow of irrigating liquid. One such device is the Aqualase® liquefaction device made and sold by Alcon, Inc. This device is designed to use heat energy to produce a pulsed flow of liquid delivered to posterior capsule 36 for liquefaction and removal of damaged or diseased lenses but can be modified in operation to be used to pulse irrigation liquid against a relatively small portion of trabecular meshwork 30 exposed by the scleral incision described above.

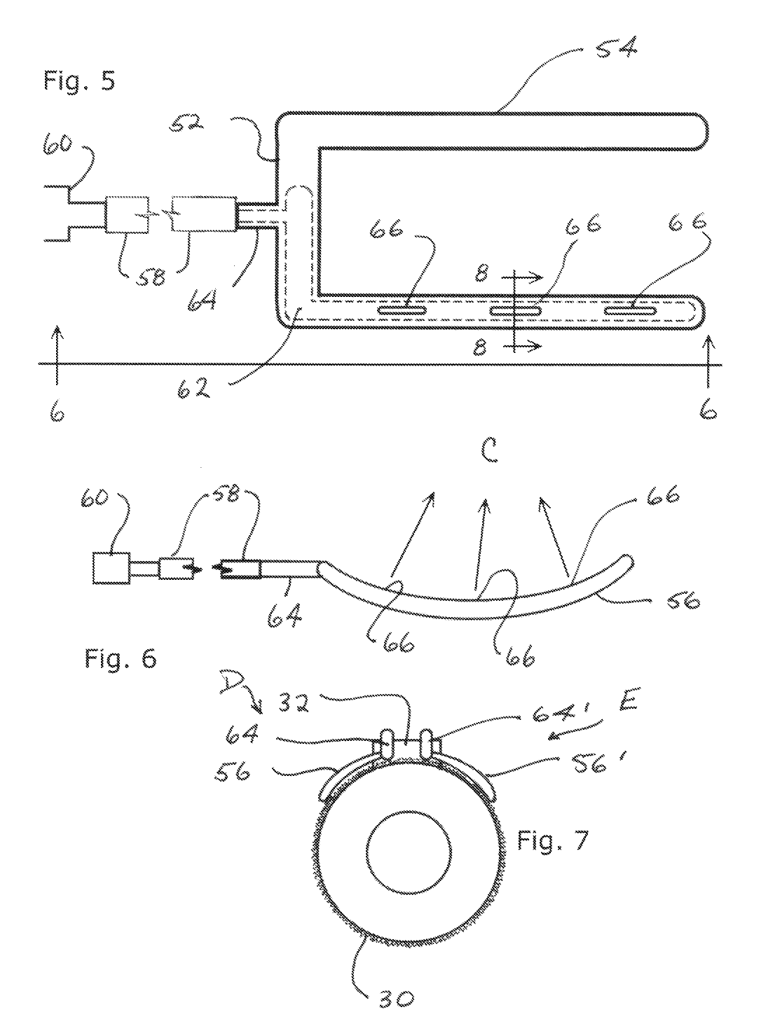

It is believed that irrigation with a non-pulsed flow of irrigating liquid also produces a desired clearing of trabecular meshwork 30. Referring to FIG. 5, the numeral 50 identifies an irrigating probe having a connecting shaft 52, an upper guide prong 54 and a lower irrigator prong 56. A length of flexible tubing 58 is attachable at one end to shaft 52 and, at the other end, to an irrigating liquid supply 60.

At least a portion of probe 50 is hollow, defining a fluid flow chamber 62 from supply 60, tubing 58, an inlet 64 formed on shaft 52, and irrigator prong 56. At least one port or, preferably, a series of ports 66 are formed along the length of irrigator prong 56 so liquid from supply 60 can be pumped, forced or pulsed through tube 58, inlet 64 and flow chamber 62 through probe 50.

As seen in FIG. 6, irrigator prong 56 is preferably curved to approximate the curve of the trabecular meshwork 30 and produces a flow pattern demonstrated by flow paths C. The curve shown in FIG. 6 will be referred to as a right-hand curve, that is, as viewed in FIG. 6 from “below” probe 50, irrigator prong 56 curves back to the right. It should be understood that a similar irrigator identified as 56′ is also provided with an opposite, or left-hand curve when viewed and described as above, and with a left-hand guide prong 54′ as well.

Referring to FIG. 7 and recalling FIG. 4A, probe 50 is shown inserted into Schlemm's canal 32 with guide prong 54 (not shown so that irrigator prong 56 can be seen more clearly) contacting cornea 14. Irrigator prong 56 is thus positioned proximate a section of trabecular meshwork 30. Irrigating liquid is then introduced from supply 60 through tubing 58 and shaft 52, entering irrigator 56 and flow chamber 62 and exiting via ports 66 to impinge against trabecular meshwork 30.

Supply 60 can, selectively, produce steady, discrete or pulsed flow of liquid at desired temperatures and pressures.

Referring now to FIG. 7 another method is illustrated to treat an increased portion of trabecular meshwork 30 using a single incision. Right-hand irrigator prong 56 is first inserted into Schlemm's canal 32 facing in direction D and liquid is introduced to treat a first portion of trabecular meshwork 30. After treatment, irrigator prong 56 is removed and left-hand irrigator prong 56′ is inserted in direction E to treat another portion of trabecular meshwork 30. It is believed that treatment of as large a portion as possible will produce better and longer-lasting therapeutic effects.

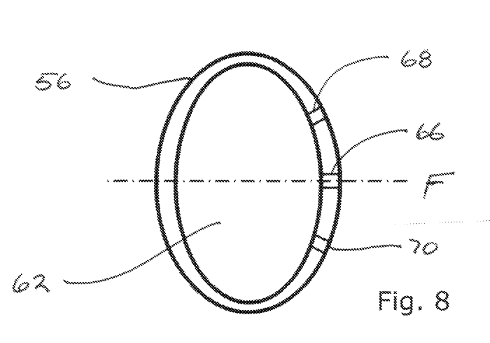

Referring now to FIG. 8, a sectional view of irrigator prong 56 is shown demonstrating adjustment of the flow pattern from flow chamber 62 by the positioning and angling of the exit ports 66. For purposes of reference, irrigator prong 56 is shown having a transverse axial plane F which bisects and follows the curve of the cross section of irrigator prong 56.

As an example, selected of ports 66 can be angled upward with respect to axial plane E as shown at port 68, set to a horizontal position as at port 66 with at least a portion of port 66 lying in axial plane F; or angled downward with respect to axial plane E as shown at port 70. Ports of various orientations and positions can be included on the same irrigator prong 56 to treat as large an expanse of trabecular meshwork 30 as possible in a variety of selected patterns.

This improved treatment allowed control of open angle glaucoma using a relatively simple surgical procedure involving no use of artificial expedients such as shunts and resulted in a patient condition that responded to topical medication, reducing the need for systemic medication

While the foregoing describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

I claim:

1. A surgical instrument for providing a flow of irrigating liquid from an irrigating liquid source to selected portions of an eye, said instrument comprising:
an irrigating probe,
said probe having an inlet for irrigating liquid;
means for connecting said inlet to said liquid source;
said probe having a guide prong having first and second ends;
said probe having an irrigator prong having first and second ends,
said guide prong arm and said irrigator prong held in fixed spatial relationship generally parallel and spaced apart from one another by a shaft extending from said first guide prong end to said first irrigator prong end,
a port formed on said shaft,
said shaft being fluid-tightly attached to said connecting means at said port,
a fluid flow channel formed through at least a portion of said shaft and said irrigator prong extending from said port through said shaft and said irrigator prong, and
at least one irrigating port formed on said irrigator prong intermediate said first and second ends,
said irrigating port communicating with said fluid flow channel.

2. The apparatus as recited in claim 1 wherein said irrigator prong is curved in a first, right hand direction with respect to said shaft.

3. The apparatus as recited in claim 1 wherein said irrigator prong is curved in a second, left hand direction with respect to said shaft.

4. The apparatus as recited in claim 1 wherein said irrigator prong has a transverse axial plane and at least a portion of one said irrigating port lies in said plane.

5. The apparatus as recited in claim 1 wherein said irrigator prong has a transverse axial plane and at least one said irrigating port is formed at an angle to said plane.

6. The apparatus as recited in claim 1 wherein said irrigator prong has two or more irrigating ports formed thereon.

7. The apparatus as recited in claim 4 wherein said irrigating prong has at least one said irrigating port having no portion thereof lying in said plane.

8. The apparatus as recited in claim 1 wherein said irrigating liquid is pulsed through said probe.

9. The apparatus as recited in claim 1 wherein said irrigating liquid is passed through said probe in a non-pulsed flow.
